(19) United States
(12) Patent Application Publication
(10) Pub. No.: US 2018/0091033 A1
(43) Pub. Date: Mar. 29, 2018

(54) In-Body Power Harvesting Using Flowing Fluids

(71) Applicant: QUALCOMM Incorporated, San Diego, CA (US)

(72) Inventor: William Henry Von Novak, III, San Diego, CA (US)

(73) Assignee: QUALCOMM Incorporated, San Diego, CA (US)

(21) Appl. No.: 15/273,894

(22) Filed: Sep. 23, 2016

(51) Int. Cl. H02K 44/08; A61N 1/378; H01F 7/02; H02K 44/16

(52) U.S. Cl. CPC H02K 44/085; H02K 44/16; H01F 7/0273; A61N 1/3785

(57) ABSTRACT

The present disclosure describes aspects of in-body power harvesting using flowing fluids. In some aspects, an apparatus comprises a magnetic field generator configured to generate a magnetic field to deflect charged particles of a fluid flowing through the body in a space formed by a vessel. The charged particles include positively and negatively charged particles, which the magnetic field deflects such that the positively charged particles are deflected in a first direction within the space and such that the negatively charged particles are deflected in a second direction within the space generally opposing the first direction. The apparatus also includes electrodes configured to harvest energy based on a difference in potential between the positively charged particles and the negatively charged particles. The harvested energy is convertible by a converter coupled to the electrodes into electrical power that is usable by an electronic device.

### FIELD OF THE DISCLOSURE

This disclosure relates generally to harvesting power within a body. More particularly, the disclosure relates to harvesting power through magnetohydrodynamic (MHD) generation.

### BACKGROUND

This description of related art is provided for the purpose of generally presenting a context for the disclosure that follows. Unless indicated otherwise herein, concepts described in this section are not prior art to this disclosure and are not admitted to be prior art by inclusion herein.

Biomedical implants are becoming more common for treatment of disease and medical conditions in humans as well as in animals. These implants can be inserted into a host's body for a variety of purposes, such as to release metered doses of medication, stimulate bodily tissue (e.g., nerves), monitor specific biochemical conditions, and so on. Oftentimes, such implants require electrical energy in order to operate—they need a power source, which typically takes the form of a chemical battery. Although implants are expected to be operative for several years (or a host's lifetime) without replacement, the chemical batteries used to power them may not be capable of operating that long. Thus, to keep these implants operating as designed, their batteries may need to be changed. Changing chemical batteries that are implanted can be difficult, however, and doing so can pose a significant risk to the host. Accordingly, conventional techniques for powering implants can put a host's life at risk.

### SUMMARY

In some aspects of in-body power harvesting using flowing fluids, an apparatus comprises a magnetic field generator configured to generate a magnetic field to deflect charged particles of a fluid flowing through the body in a space formed by a vessel. The charged particles include positively and negatively charged particles, which the magnetic field deflects such that the positively charged particles are deflected in a first direction within the space and such that the negatively charged particles are deflected in a second direction within the space generally opposing the first direction. The apparatus also includes electrodes configured to harvest energy based on a difference in potential between the positively charged particles and the negatively charged particles. The harvested energy is convertible by a converter coupled to the electrodes into electrical power that is usable by an electronic device.

Some aspects of in-body power harvesting using flowing fluids also involve a method in which a magnetic field is generated to deflect charged particles of a fluid flowing through a space in a body. The charged particles include positively and negatively charged particles, which the magnetic field deflects in generally opposing directions. The method also comprises harvesting energy from the flowing fluid via electrodes. The electrodes harvest the energy based on a difference in potential between a portion of the fluid with the positively charged particles and a portion of the fluid with the negatively charged particles. Further, the method includes converting the harvested energy with a converter coupled to the electrodes into electrical power that is usable by an electronic device.

In other aspects, an apparatus for harvesting power from a body comprises two stacked disk-shaped circular containers centered substantially around a shared longitudinal axis. The apparatus also includes two tubes that are arranged on opposing sides of the circular containers and are configured to feed fluid from the body into a respective circular container. Further, magnetic field generators of the apparatus are configured to generate magnetic fields that deflect positively and negatively charged particles of the fluid flowing through the tubes into generally opposing portions of space within the circular containers. The apparatus also includes inner and outer circular electrodes that are integral with the circular containers. These electrodes are configured to harvest energy based on a difference in potential between the positively charged particles and the negatively charged particles in the generally opposing portions of the space.

In aspects, a method for harvesting power from fluid flowing in a body comprises rerouting the fluid into tubes on opposing sides of a disk generator. In particular, the tubes feed respective disk-shaped circular containers of the disk generator. The method also includes generating magnetic fields in the tubes to deflect positively and negatively charged particles of the fluid to generally opposing portions of space in the respective circular containers. Based on a difference in potential between the positively and negatively charged particles in the generally opposing portions of the space, electrodes that circle the circular container can be used to harvest energy. Further, the method includes converting the harvested energy to electrical power that is usable by an electronic device.

In some aspects, an apparatus for harvesting power within a body using flowing fluids includes a means for generating a magnetic field that deflects charged particles of a fluid flowing in a space through the body. The charged particles include positively and negatively charged particles, which the magnetic field deflects such that the positively charged particles are deflected in a first direction within the space and such that the negatively charged particles are deflected in a second direction within the space generally opposing the first direction. The apparatus also includes a means for harvesting energy based on a difference in potential between the positively charged particles and the negatively charged particles. To enable an implant in the body to use the electrical power, the apparatus may also include a means for converting harvested power into a form that is usable by the implant.

### BRIEF DESCRIPTION OF DRAWINGS

The details of various aspects are set forth in the accompanying figures and the detailed description that follows. In the figures, the left-most digit of a reference number identifies the figure in which the reference number first appears. The use of the same reference numbers in different instances in the description or the figures indicates like elements:

FIG. 1 illustrates an example environment that includes an in-body power-harvesting device.

FIG. 2 illustrates an example configuration of the in-body power-harvesting device shown in FIG. 1.

FIG. 3 illustrates two example arrangements of electrodes along a vessel for an in-body power harvesting device.

FIG. 4 illustrates another example configuration of an in-body power-harvesting device.

FIG. 5 illustrates an example method for harvesting power from fluid flowing within a body using magnetohydrodynamic (MHD) generation.

FIG. 6 illustrates an example method for harvesting power from fluid flowing within a body using a disk generator.

FIG. 7 illustrates a system-on-chip (SoC) having components through which aspects of in-body power harvesting using flowing fluids can be implemented.

### DETAILED DESCRIPTION

Devices implanted in humans and animals are becoming more common, such as biomedical implants capable of treating disease and medical conditions. As used herein, a “host” refers to a respective body (e.g., human or animal) in which an implant is surgically inserted. Biomedical implants can be inserted into a host's body for a variety of purposes, such as to release metered doses of medication, stimulate bodily tissue (e.g., nerves), monitor specific biochemical conditions, and so on. Many implants (biomedical or otherwise) often require electrical energy in order to operate. In other words, these implants need a power source. Often the power source used to power an implant is a chemical battery. Broadly speaking, implants are capable of operating for several years (or a host's entire lifetime) without replacement. The chemical batteries used to power these implants, however, often are not capable of providing power that long. Thus, to keep an implant operating as designed, its battery may need to be surgically changed. The surgical procedures for changing implanted chemical batteries can be invasive and difficult to perform, however. Furthermore, doing so can pose a significant risk to the host. Accordingly, conventional techniques for powering implants can put a host's life at risk.

This disclosure describes aspects of harvesting power from a body (e.g., human or animal) using flowing fluids. The apparatuses and methods described herein may utilize the power harvested from the body to charge an implant's battery. Magnetohydrodynamic (MHD) generation may be used to provide a small, consistent amount of power to implants. MHD generation leverages energy in a flowing ionic fluid to generate electrical energy. To leverage this energy, a magnetic field can be used to deflect charged particles via Lorentz forces (or based on the Hall effect) toward a top and a bottom of a vessel through which the fluid flows, e.g., blood vessel, lymph vessel, urinary system tube, and so on. As used herein, a “vessel” refers to a tube within the host through which a bodily fluid flows. A vessel can be a natural tube (e.g., an artery or vein), an artificial one (e.g., a stent a shunt), or some combination thereof.

The deflection of charged particles due to the magnetic field causes a change in average charge on the top and bottom of the vessel. Using blood as an example, the magnetic field may deflect negatively charged particles flowing through a vessel toward a top inner surface of the vessel and positively charged particles toward a bottom inner surface of the vessel. As used herein the terms “top” and “bottom” simply refer to generally opposing sides of a vessel, e.g., a “top” of a blood vessel running down a person's leg from hip to foot may be the side of the vessel corresponding to the front of the person's leg, thereby making the side of the vessel that corresponds to the back of the person's leg the “bottom.” Conversely, the top of the blood vessel may correspond to the back of the person's leg and the bottom of the blood vessel may correspond to the front.

Continuing with the discussion of the change in average charge, the change corresponds to a change in concentration within the vessel of positive ions at the bottom and negative ions at the top. This change in concentration causes a potential difference between the top and bottom of the vessel. Conductive electrodes can be used to harvest this potential difference, which can drive current in an electrical circuit. Moreover, the harvested potential difference can drive current in an electrical circuit to power one or more implanted devices.

In some aspects of in-body power harvesting using flowing fluids, the magnetic field, which causes the deflection of the positive and negative ions in the flowing fluids, is produced by one or more magnets. When implanted, these magnets may be surgically positioned, as described in more detail below, relative to vessels in a way that produces the magnetic field, e.g., solenoids are arranged along generally opposing sides of a vessel. The conductive electrodes used to harvest the potential difference may be positioned as described below based on the magnetic field. For instance, the conductive electrodes may be attached to an outer surface of the vessel at a location so that the change in concentration of positive and negative ions due to the magnetic field can be leveraged to harvest power. In some aspects, the magnets and conductive electrodes are part of a disk generator into which flowing fluid may be fed on two opposing sides, and into substantially identical flattened disk-shaped circular containers, from which the flowing fluid exits along rings at the edge of the circular containers.

These and other aspects of in-body power harvesting using flowing fluids are described below in the context of an example environment, example in-body power harvesting devices, and techniques. Any reference made with respect to the example environment or power harvesting device, or elements thereof, is by way of example only and is not intended to limit any of the aspects described herein.

### Example Environment

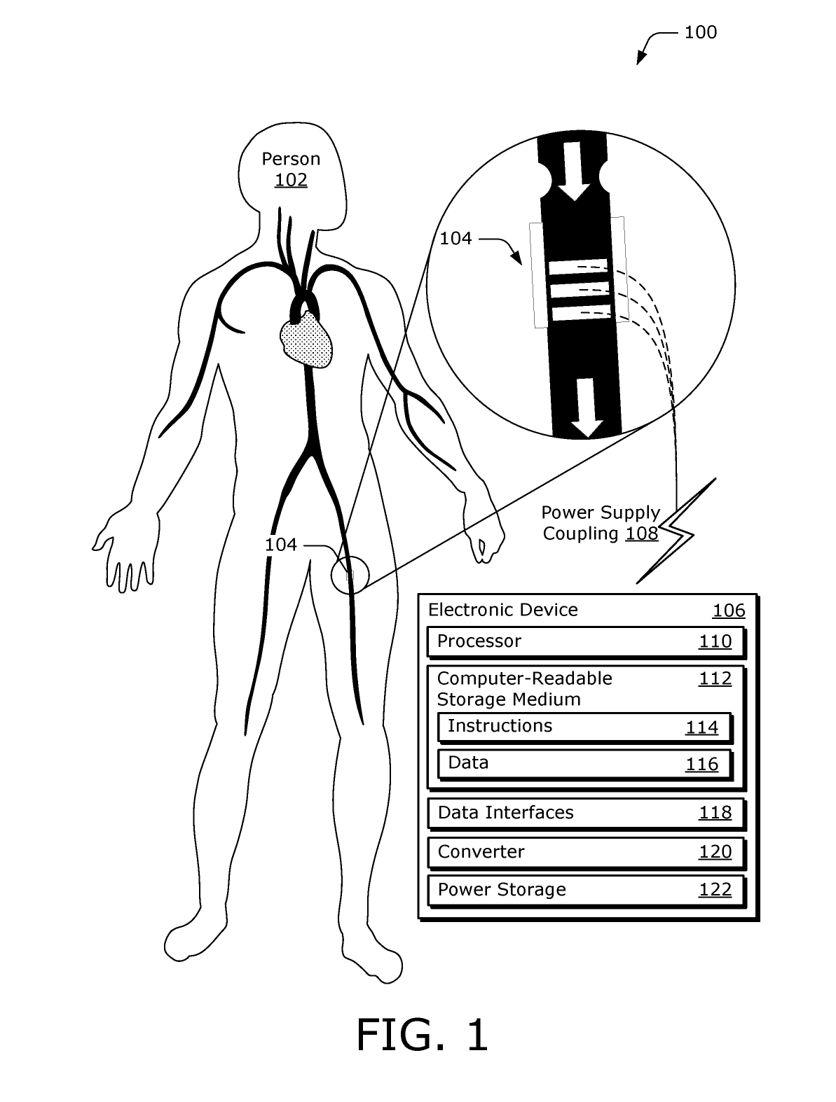

FIG. 1 illustrates an example environment 100, which includes a person 102 in which an in-body power harvesting device 104 (power harvester 104) has been implanted. In this example, electronic device 106 is also implanted in the person 102. The power harvester 104 is coupled to the electronic device 106 via power supply coupling 108 to supply the electronic device 106 with power. The electronic device 106 may be implemented as any suitable computing or electronic device that is implanted in the person 102 and capable of being powered with power harvested by the power harvester 104. Examples of the electronic device 106 include implants to release metered doses of medication, implants to stimulate bodily tissue (e.g., nerves), implants to monitor specific biochemical conditions, and so on. Electronic devices other than medical-based implants may also be contemplated within the techniques described herein, such as personal communication devices, identification devices, location tracking devices, and so forth. Accordingly, the electronic device 106 may correspond to a variety of different implanted computing or electronic devices without departing from the spirit or scope of the techniques described herein.

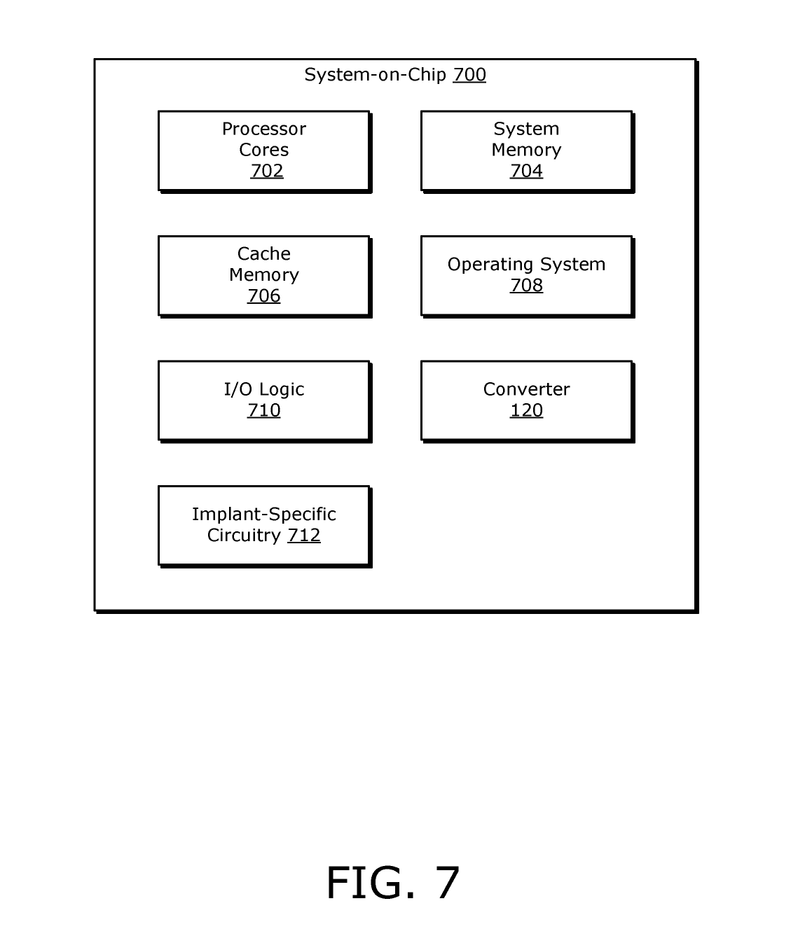

The electronic device 106 includes a processor 110. In the example, the electronic device 106 also includes computer-readable storage medium 112 (CRM 112). The processor 110 may include any type of processor, such as an application processor or multi-core processor, configured to execute processor-executable code stored by the CRM 112. The CRM 112 may include any suitable type of data storage media, such as volatile memory (e.g., random access memory (RAM)), non-volatile memory (e.g., Flash memory), optical media, magnetic media (e.g., disk or tape), and the like. In the context of this disclosure, the CRM 112 is implemented to store instructions 114, data 116, and other information of the electronic device 106, and thus does not include transitory propagating signals or carrier waves. Further, although the electronic device 106 is illustrated with the CRM 112, in some aspects the electronic device 106 may instead or additionally be implemented using a system-on-chip (SoC) as further described in relation to FIG. 7.

In the example, the electronic device 106 also includes data interfaces 118. The data interfaces 118 provide connectivity to respective networks and other electronic devices connected therewith. The data interfaces 118 may comprise wired data interfaces (that are usable to connect with the electronic device 106 before it is implanted into a body, during a surgical procedure in which the electronic device 106 is exposed, when the electronic device 106 has been removed from the body, and so on), wireless data interfaces, or any suitable combination thereof. Alternately or additionally, the wireless interfaces may include a modem or radio configured to communicate over a wireless network, such as a wireless LAN, peer-to-peer (P2P), cellular network, and/or wireless personal-area-network (WPAN).

The electronic device 106 also includes converter 120 and power storage 122. The converter 120 converts power received from the power harvester 104 via the power supply coupling 108. The converter 120 converts the power into a form that is usable by the electronic device 106 to perform its corresponding functionality, e.g., release metered doses of medication, stimulate bodily tissue (e.g., nerves), monitor specific biochemical conditions, and so on. By way of example, the converter 120 represents functionality to boost a voltage of the power harvested. The converter 120 may be used to convert a voltage of the harvested power that is lower than what the electronic device 106 needs to perform its corresponding functionality. Although the converter 120 is illustrated as part of the electronic device 106 in the example, in some aspects the converter 120 may be separate from the electronic device 106 and coupled thereto. The converter 120 may, for instance, be inserted into the power supply coupling 108 between the power harvester 104 and the electronic device 106. In this scenario, unboosted, harvested power flows from the power harvester 104 via a first portion of the power supply coupling 108 to the converter 120, and power having a boosted voltage flows from the converter 120 via a second portion of the power supply coupling 108 to the electronic device 106.

In some scenarios, the power harvester 104 may harvest more power than is usable by the electronic device 106 at the time. To store this excess power, the power storage 122 may be used. The power storage 122 represents functionality to store power received via the power supply coupling 108 for later use. For instance, the power storage 122 may be configured as a type of battery. In some aspects, the converter 120 may feed the power storage 122, and the electronic device 106 may draw power for operation from the power storage 122. In other aspects, the electronic device 106 may draw power for operation directly from the converter 120 and rely on the power storage 122 solely when the power supplied directly from the converter 120 is not enough to function properly. In both cases, the electronic device 106 is configured to use power stored in the power storage 122 for operation.

Regardless of a configuration or function of the electronic device 106, the power harvester 104 supplies the electronic device 106 with power via the power supply coupling 108. In some aspects, the power supply coupling 108 is a wired configuration in which conductive electrodes of the power harvester 104 are connected to the electronic device 106 using a set of wires. In other aspects, the power supply coupling 108 is a wireless configuration in which a power transfer unit connected to the power harvester 104 enables wireless transfer of power to a power receiving unit connected to the electronic device 106. The power supply coupling 108 may be configured in a variety of ways to transfer harvested power from the power harvester 104 to the electronic device 106 without departing from the spirit or scope of the techniques described herein.

In general, the power harvester 104 represents functionality to harvest power from fluid flowing through a vessel of the person 102. The power harvester 104 may be configured as an apparatus and/or multiple interoperable components that are surgically implanted into the person 102 and capable of using magnetohydrodynamic (MHD) generation to provide a small, consistent amount of power. To do so, the power harvester 104 generates a magnetic field through which a fluid in a vessel of the person 102 passes, e.g., blood flowing through a blood vessel, urine or other liquids flowing through a tube of the urinary system, lymph flowing through a lymphatic vessel, cerebrospinal fluid flowing through ventricles, and so on. The magnetic field deflects positively charged ions of the fluid and negatively charged ions of the fluid in generally opposing directions. In some aspects, for instance, the magnetic field deflects negatively charged ions of the fluid toward a top of the vessel and positively charged ions toward a bottom. The separation of positively and negatively charged ions in the fluid results in potential energy across the fluid, e.g., between the top and bottom of the vessel.

The power harvester 104 is also configured with conductive electrodes capable of harvesting this potential energy. For instance, sets of the electrodes may be positioned along generally opposing sides of an outer surface of the vessel and downstream from the magnetic field, at a location where the positive ions and negative ions are generally separated. Additionally, the set of electrodes on one side of the vessel may be connected to each other forming one end of a circuit. Another set of electrodes on the opposing side of the vessel may be connected to each other forming another end of the circuit. When the separated ionic fluid flows past the electrodes, it is causes a current to flow in the circuit. The produced current may thus serve as a basis for powering the electronic device 106.

Although the power harvester 104 is shown powering a device that is also implanted in the person 102, it should be appreciated that the power harvested by the power harvester 104 may be used to power devices outside of the person 102. As mentioned above, the power supply coupling 108 may be wirelessly implemented. As such, a device with a power receiving unit outside the person 102 may be capable of receiving power wirelessly from a power transfer unit located inside the person 102 and connected to the power harvester 104. By way of example, power harvested by the power harvester 104 may be used to power a watch worn by the person 102, a mobile device in the person 102's pocket, and so on. How a power harvester 104 may be specifically implemented to harvest power from flowing fluids in a body is described in greater detail below.

### Example In-Body Power Harvesters

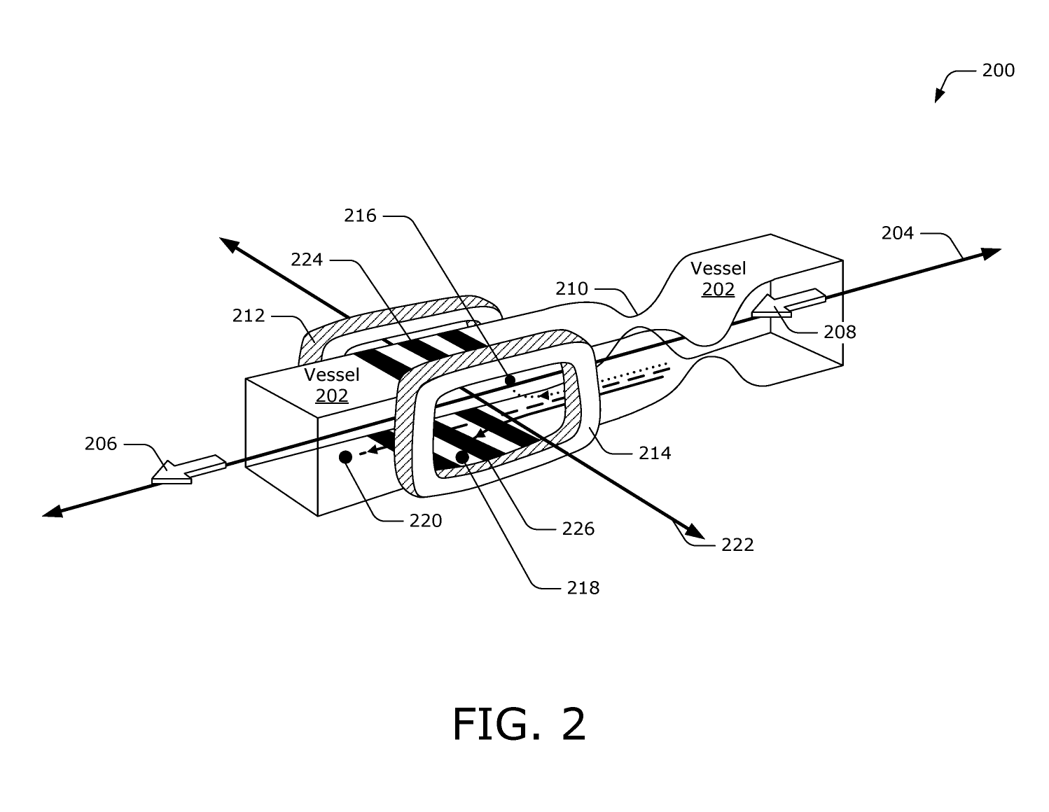

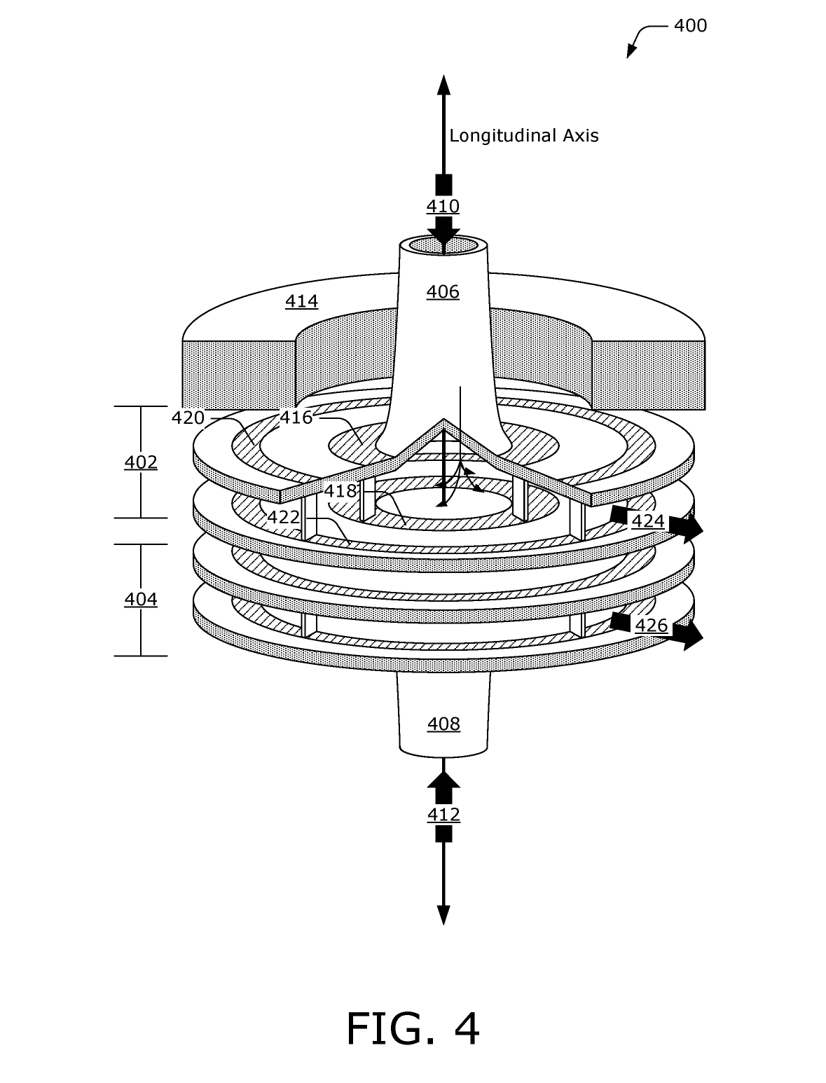

FIG. 2 illustrates an example in-body power harvesting device 104 (power harvester 104) for harvesting power from flowing fluids in accordance with one or more aspects at 200. In particular, the power harvester 104 illustrated at 200, and similar implementations, may be referred to as a “nozzle configuration.” In contrast to the “nozzle configuration” of FIG. 2, a “disk generator configuration” may also be used and is described in detail with reference to FIG. 4.

In the nozzle configuration, components of the power harvester 104 are arranged in relation to vessel 202. The vessel 202 may be a naturally occurring tube within a body, such as blood vessel, a tube of the urinary system, a lymphatic vessel, and so on. Thus, although the vessel 202 is illustrated as being generally rectangular in shape, this is simply for the convenience of discussing how the components of the power harvester 104 are arranged relative to the vessel 202. Instead, the vessel 202 may be a tube that is generally cylindrical in shape. Since the vessel 202 may correspond to a naturally occurring tube in a body, it should be appreciated that a shape of the vessel may vary accordingly. In yet other aspects, the vessel 202 may be artificial. The vessel 202 may be implanted into the body along with the power harvester 104, for instance, and may correspond to any of a variety of implantable tubes through which fluids of the body can flow, such as a stent, a shunt, and so on.

In aspects, the vessel 202 extends generally along major axis 204. The vessel 202 also has an outer surface, which defines a volume within the vessel 202—a space through which a corresponding fluid or fluids can flow. When the vessel 202 is an artery, for instance, the outer surface corresponds to artery walls that form a space through which blood can flow. The fluid flowing through the vessel 202 flows in a direction generally along the major axis 204. Arrows 206, 208 illustrate a general direction in which a corresponding fluid flows through the vessel 202.

In the illustrated example, the vessel 202 also has constriction 210. In some aspects, the constriction 210 can be natural, e.g., a urethral sphincter. In other aspects, the constriction 210 may be artificial, e.g., the vessel 202 may be constricted by surgically removing some of the outer surface, placing a band (not shown) around the vessel 202 to cause the constriction 210, and so forth. In general, the constriction 210 represents a portion of the vessel 202 that is narrower, or made narrower, than the preceding and succeeding portions of the vessel 202. Due to the narrowed passageway, the constriction 210 causes acceleration of fluid flowing through the vessel 202. With regard particularly to harvesting energy, the faster the fluid flows, the more power the power harvester 104 can harvest. Although some of the configurations discussed herein may include a constriction (natural or artificial), some configurations may not have such a constriction. By way of example, where a velocity of fluid flowing through an unconstricted vessel is sufficient for harvesting power, a constriction may not be added.

In the illustrated example, the power harvester 104 includes magnets 212, 214. Although two magnets are illustrated, the power harvester 104 may be configured with fewer magnets (at least one) or more magnets (three or more) without departing from the spirit or scope of the techniques described herein. In general, the magnets 212, 214 represent functionality of the power harvester 104 to generate a magnetic field. The magnets 212, 214 may thus serve as a “magnetic field generator,” which refers to components capable of generating a magnetic field. In this example, the magnetic field extends through the outer surface of the vessel 202 as well as the space within, for at least some length along the major axis 204. In any case, the fluid flowing through the vessel 202 passes through the generated magnetic field.

When the fluid flows through the magnetic field, charged particles are deflected via Lorentz forces (or based on the Hall effect) to generally opposing sides of the space within the vessel. To illustrate this, negative ion 216 and positive ion 218 are included in the example at 200. The negative ion 216 is illustrated along a path (the dotted particle line), which indicates that the negative ion 216 has been deflected from an original course toward a top of the vessel 202. In contrast, the positive ion 218 is illustrated along another path (the solid particle line), which indicates that the positive ion 218 has been deflected from an original course toward a bottom of the vessel 202. A third ion 220 is illustrated passing straight through the vessel (with the dashed particle path). This may be the case for some particles passing through a center of the vessel, e.g., a minority of particles that the magnetic field is not capable of deflecting, that are blocked from being deflected by other particles, and so forth.

In any case, the magnetic field deflects negatively charged particles of the fluid generally in one direction and positively charged particles of the fluid in an opposing direction. In some aspects, the magnets 212, 214 correspond to solenoids, although other types of magnets and configurations, capable of generating a magnetic field are also contemplated. By way of example, the power harvester 104 may be configured with permanent magnets, U-shaped magnets, and/or “E” core magnets. The magnetic field may be created using still other types of magnets or components capable of generating the magnetic field in the spirit and scope of the technique described herein.

In the illustrated example, the magnets 212, 214 are arranged relative to each other in a spaced relationship, on generally opposing sides of the vessel 202. The space between the magnets 212, 214 corresponds to a width of the vessel. Further, the example includes orthogonal axis 222, which is generally orthogonal to the major axis 204. In accordance with one or more aspects, the magnets 212, 214 are arranged on generally opposing sides of the outer surface of the vessel 202, and are generally centered around the orthogonal axis 222. The magnets 212, 214 may be positioned in this way through a surgical procedure that attaches the magnets to the vessel 202. When the vessel 202 itself is artificial, the magnets 212, 214 may be mounted to the outer surface of the vessel 202 as part of a process to manufacture the power harvester 104. In other nozzle configurations, a pair of magnets may be arranged in different ways with respect to the vessel 202.

In accordance with one or more aspects, deflecting the positive ions of the flowing fluid in one direction and the negative ions in a generally opposing direction creates electric potential across the fluid. The example at 200 includes sets of electrodes 224, 226, which represent conductive electrodes and functionality of the power harvester 104 to harvest power by leveraging this electric potential. In aspects, such the one illustrated in FIG. 2, the sets of electrodes 224, 226 are arranged on generally opposing sides of the outer surface of the vessel 202. Further, the sides of the vessel 202 where the electrodes are arranged are between the generally opposing sides where the magnets 212, 214 are arranged. Assuming for the purpose of discussion, that the vessel 202 is rectangular in shape and that the magnets 212, 214 are disposed on a right and left side of the vessel 202, then the electrodes are arranged on a top and bottom of the vessel 202. To this extent, the sets of electrodes 224, 226 have a spaced relationship in which the sets are separated by the vessel 202.

In some aspects, the sets of electrodes 224, 226 lie on portions of the vessel 202's outer surface that are across from one another along an axis orthogonal to both the major axis 204 and the orthogonal axis 222. With regard further to position, the sets of electrodes 224, 226 are arranged to leverage the potential between the separated negative and positive ions. In some aspects, this position may be just downstream of the magnets 212, 214. In other aspects, this position may be within a length of the major axis 204 that begins at a most upstream portion of the magnets 212, 214 and ends at a most downstream portion of the magnets 212, 214. When arranged within this length, the position may nevertheless be biased downstream in relation to a center of the magnets 212, 214.

A given set of electrodes may include multiple electrodes in a segmented arrangement. In this arrangement, the electrodes are disposed along the outer surface of the vessel 202, such that there is space between each electrode of the set. The space between the electrodes may be substantially uniform in some aspects, and may vary in others. Although not shown, wires may connect the sets of electrodes 224, 226, such that each electrode of the set of electrodes 224 is connected with the other electrodes of the set forming one end of a circuit, and such that each electrode of the set of electrodes 226 is connected with the other electrodes of the set forming the other end of the circuit. The separated charged particles (negative ions drawn generally to one side of the vessel and positive to the other) flowing past the electrodes cause current to flow through the wires. In this way, the sets of electrodes 224, 226 can generate power sufficient to power an implant, such as the electronic device 106. Manners in which the sets of electrodes 224, 226 are connected may be varied as discussed below.

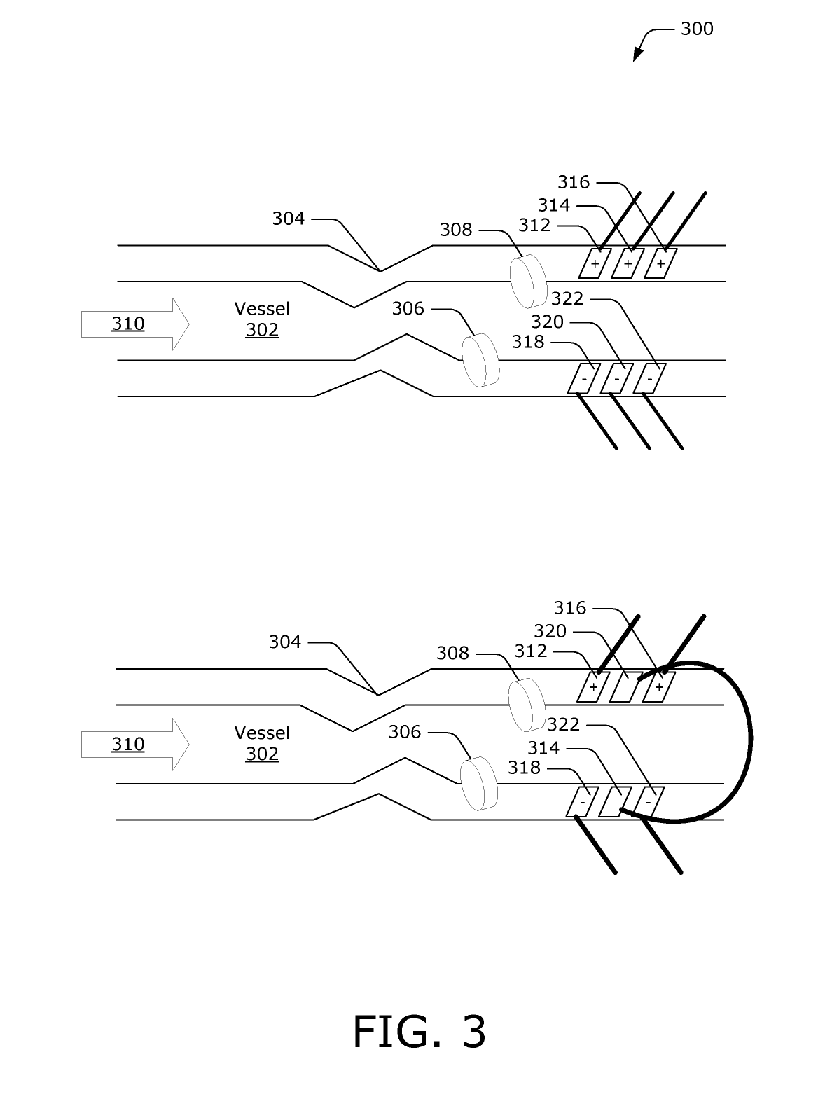

FIG. 3 illustrates two example arrangements of electrodes along a vessel for an in-body power harvesting device in accordance with one or more aspects at 300. Both examples are described with reference to vessel 302, having constriction 304, and include magnets 306, 308. Further, both examples include arrow 310, which indicates a direction fluid flows within the vessel 302. By way of example, the vessel 302 may correspond to a blood vessel (vein or artery) and the constriction 304 may be artificial, e.g., caused by a ring (not shown) placed around the vessel 302 as part of a surgical procedure. In this scenario, the fluid flowing in the direction of the arrow 310 is blood.

In the first (top) arrangement example of FIG. 3, positive electrodes 312, 314, 316 are arranged along a wall on one side of the vessel, and negative electrodes 318, 320, 322 are arranged along a wall on a generally opposing side of the vessel. In this example arrangement, lines representing wires extend from each of the electrodes 312, 314, 316, 318, 320, 322. This example arrangement thus represents a scenario in which the electrodes on one side of the vessel 302 are each positive, and the electrodes on the generally opposing side are each negative. This example arrangement further represents a scenario in which the positive electrodes 312, 314, 316 are wired together to form one end of an electrical circuit, and the negative electrodes 318, 320, 322 are wired together to form another end of the electrical circuit. Accordingly, each of the electrodes functions as a harvesting electrode.

The second (bottom) arrangement example of FIG. 3, represents an alternative arrangement of electrodes. In this example arrangement, the positive electrodes 312, 316 are arranged along the wall of the one side of the vessel, and the negative electrodes 318, 322 are arranged along the wall of the generally opposing side of the vessel 302, similar to the first arrangement example. In contrast to the first example, however, the center electrodes (e.g., the negative electrode 320 and the positive electrode 314) may also be connected to each other, as illustrated in the example. This electrically connection together causes the electrodes to have a same potential. Connecting the center electrodes in this way provides an alternate path for the current thereby increasing the efficiency of the circuit. In arrangements similar to this second example electrode arrangement (e.g., having three electrodes on each side of the vessel), the leftmost and rightmost electrodes function as harvesting electrodes. Rather than using nozzle configurations as discussed in reference to FIGS. 2-3, the power harvester 104 may be configured in other ways without departing from the spirit or scope of the described techniques.

FIG. 4 illustrates another example in-body power harvesting device 104 (power harvester 104) for harvesting power from flowing fluids in accordance with one or more aspects at 400. In particular, the example, generally at 400, depicts a disk generator.

The disk generator includes two flattened, disk-shaped circular containers 402, 404 (circular containers 402, 404). The circular containers 402, 404 may have a stacked relationship in which they are substantially centered around a shared longitudinal axis. An ionic fluid of the body is fed into two opposing sides of the implanted disk generator. In particular, the fluid is fed through tubes 406, 408 and into the corresponding circular containers 402, 404. A major axis of each tube may align substantially with the shared longitudinal axis. Fluid fed into the disk generator through the tube 406 passes through the circular container 402, and fluid fed into the disk generator through the tube 408 passes through the circular container 404. Arrows 410, 412 indicate the direction fluid is fed into the tubes 406, 408, respectively.

The disk generator also includes magnet 414. Although the example at 400 includes just the magnet 414 positioned around the tube 406, in implementation a matching magnet may be positioned around the tube 408. These magnets may be permanent magnets, electromagnets, and so forth. Regardless of how implemented, the magnet 414 represents functionality of the disk generator to create magnetic fields for the fluid flowing through the tubes 406, 408. Like the nozzle configuration, these magnetic fields cause the positive and negative ions of the fluid to separate.

In aspects, the disk generator includes inner electrodes 416, 418 that circle the circular container 402 substantially where the fluid enters. The disk generator also includes outer electrodes 420, 422 that circle the circular container 402 substantially where the fluid exits. The inner and outer electrodes may also be centered substantially around the shared longitudinal axis. In aspects, these electrodes are integral with the circular container 402. Arrows 424, 426 indicate the direction the fluid exits the circular containers 402, 404, respectively. Broadly speaking, the fluid flows into the disk generator through the tubes 406, 408 on the two opposing sides, is deflected at a right angle, and exits along a ring at the edge of the circular containers 402, 404. The fluid thus exits the disk generator in a direction that is substantially perpendicular to a direction the fluid enters. Although electrodes of the circular container 404 are not labeled, the circular container 404 may be configured with inner and outer electrodes similar to those of the circular container 402.

These electrodes are configured to harvest the potential energy caused by separating the positive and negative ions of the fluid flowing into the disk generator. As the fluid enters the disk generator through the tubes 406, 408, ionic components of the fluid are deflected at a right angle to the magnetic fields created by the magnet 414 and a magnet around the tube 408. Although the deflection of the ionic components may differ based on a charge of the ion and a direction of the magnetic field through which the ion flows, in the illustrated example, positive ions may be driven outward toward the outer electrodes 420, 422 and negative ions driven inward toward the inner electrodes 416, 418. The difference in charge between the ions driven inward and the ions driven outward creates electric potential. This potential can be harvested by the electrodes 416, 418, 420, 422, thereby generating power for a circuit. Accordingly, the power generated by the disk generator can be used to power the electronic device 106.

In some aspects, a disk generator like the one illustrated in FIG. 4 may be surgically implanted into a body, such as into the person 102. The surgical procedure for implanting such a disk generator may involve rerouting the flow of fluid through the body. In particular, the fluid may be rerouted to enter the disk generator on two sides and exit from the disk generator in a substantially perpendicular direction. This may be accomplished by attaching shunts to naturally occurring vessels in the body, causing the fluid to flow from those vessels through the disk generator. Additional shunts may be used to reconnect to a portion of the vessel downstream. In this way, the flow of fluid through the body is merely temporarily diverted—the fluid is returned to flow along its normal course.

### Techniques of In-Body Power Harvesting Using Flowing Fluids

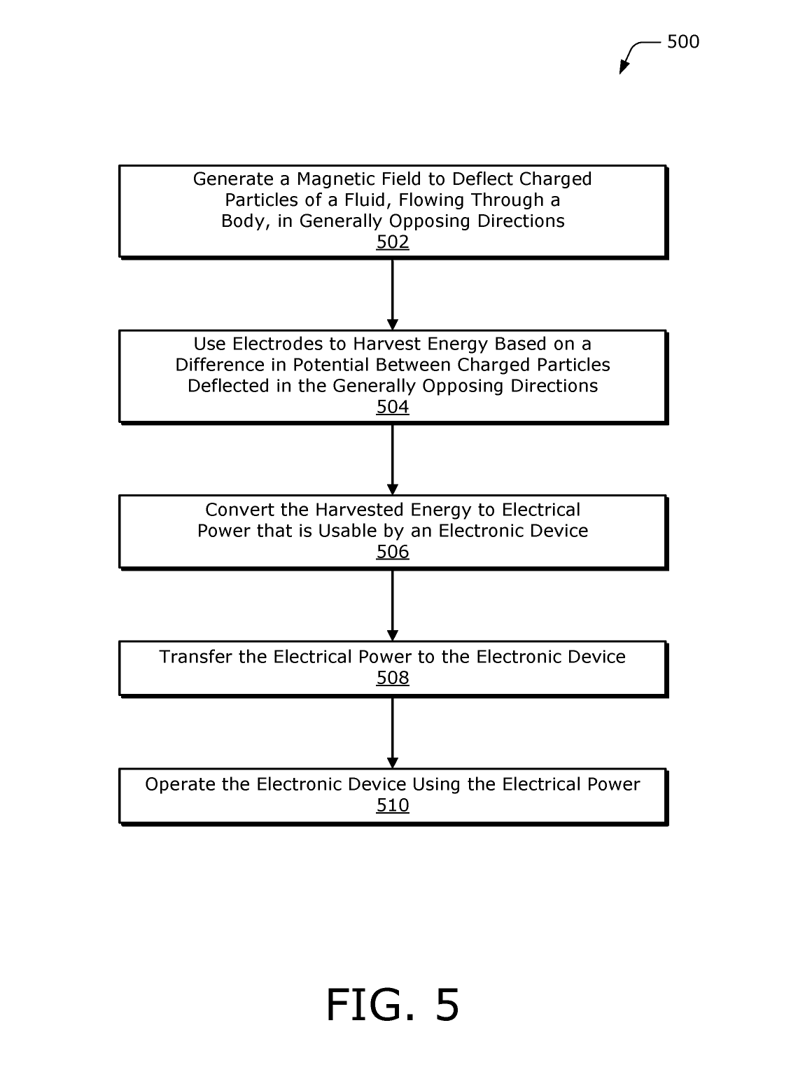

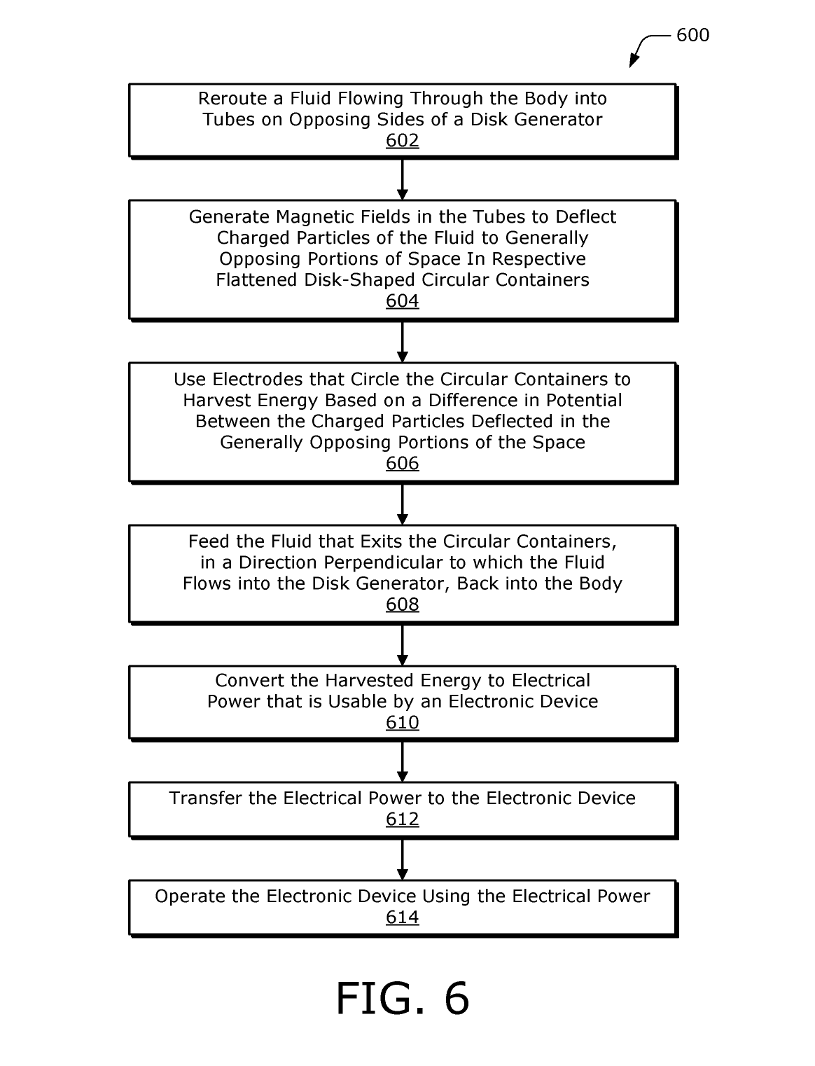

The following techniques of in-body power harvesting using flowing fluids may be implemented using any of the previously described power harvesters of the example environment. The techniques may also involve powering an implant configured like the electronic device 106 of the example environment or the system-on-chip described with reference to FIG. 7. Reference to entities, such as the power harvester 104 or the electronic device 106, is made by example only and is not intended to limit the ways in which the techniques can be implemented. The techniques are described with reference to example methods illustrated in FIGS. 5 and 6. The example methods are depicted as respective sets of operations or acts that may be performed using the entities described herein and/or any suitable components which provide means for implementing one or more of the operations. The depicted sets of operations illustrate a few of the many ways in which the techniques may be implemented. As such, operations of a method may be repeated, combined, separated, omitted, performed in alternate orders, performed concurrently, or used in conjunction with another method or operations thereof

FIG. 5 illustrates an example method 500 of in-body power harvesting from flowing fluid using magnetohydrodynamic (MHD) generation, including operations performed by the power harvester 104. In the following discussion, the power harvester 104 or other entities of example environment 100 may provide means for implementing one or more of the operations described.

At 502, the method includes generating a magnetic field by a magnetic field generator to deflect charged particles of a fluid, flowing through the body, in generally opposing directions. The fluid flows through a space in the body and the charged particles include positively and negatively charged particles. The magnetic field deflects the positively charged particles in a first direction within the space and deflects the negatively charged particles in a second direction within the space that generally opposes the first direction.

By way of example, consider FIG. 2, which illustrates a nozzle configuration of the power harvester 104 in accordance with one or more aspects generally at 200. In this example, the magnets 212, 214 serve as a magnetic field generator and generate a magnetic field in a space enclosed by walls of the vessel 202. The fluid generally flows through the space within the vessel along the major axis 204 and in the direction indicated by the arrows 206, 208. When the fluid passes through the generated magnetic field, positively charged particles of the fluid are deflected in a first direction within the vessel 202. The magnets 212, 214 also deflect negatively charged particles of the fluid in a second direction within the vessel 202 that is generally opposed to the first direction.

This deflection in generally opposing directions is illustrated in FIG. 2 by the negative ion 216, the positive ion 218, and their respective paths through the vessel 202. In particular, the corresponding path of the negative ion 216 indicates deflection upward, toward a top wall of the vessel 202. Additionally, the corresponding path of the positive ion 218 indicates deflection downward, toward a bottom wall of the vessel 202. Deflecting positively charged particles generally in one direction and negatively charged particles generally in an opposing direction causes the flowing fluid to have a higher concentration of the positively charged particles flowing on one side of the space and a higher concentration of the negatively charged particles flowing on the generally opposing side. This separation of negatively and positively charged flowing particles creates potential energy across the vessel 202, e.g., between the portion of the fluid generally having the negatively charged particles and the portion of the fluid generally having the positively charged particles.

At 504, the method includes using electrodes to harvest energy based on a difference in potential between the charged particles deflected in the generally opposing directions. By way of example, the sets of electrodes 224, 226 are used to harvest energy from the fluid flowing through the vessel 202 past the magnetic field generated by the magnets 212, 214. In particular, the sets of electrodes 224, 226 harvest energy based on the above-discussed difference in potential created by deflecting the negatively and positively charged to generally opposing sides within the vessel 202. Further, the electrodes of a given set may be connected with each other forming respective ends of a circuit, as in the first (top) example depicted in FIG. 3. The separated fluid, flowing past the sets of electrodes 224, 226 causes current to flow in the circuit. In other words, the energy is harvested.

At 506, the method includes converting the harvested energy to electrical power that is usable by an electronic device. By way of example, the converter 120 converts the energy harvested by the power harvester 104 to electrical power that is usable by the electronic device 106. In some aspects, the converter 120 may change (e.g., increase) a voltage of the harvested energy to match a voltage used by the electronic device 106. The converter 120 may also convert the harvested energy in other ways such as converting the current from one form to another, e.g., converting direct current (DC) to alternating current (AC), converting AC to DC, and so on. The converter 120 may convert the harvested energy in still other ways without departing from the spirit or scope of the techniques described herein.

At 508, the method includes transferring the electrical power to the electronic device. By way of example, the power is transferred from the power harvester 104 to the electronic device 106 via the power supply coupling 108. As discussed above, the power supply coupling 108 may be configured as a wired or wireless connection. As such, the electrical power may be transferred across a wired connection from the power harvester 104 to the electronic device 106. Alternately, the electrical power may be transferred wirelessly from the power harvester 104 to the electronic device 106. A wireless implementation provides the advantage of being able to power electronic devices outside of a body (e.g., of the person 102) using power generated within the body.

At 510, the method includes operating the electronic device using the electrical power. By way of example, the electronic device 106 carries out the functionality for which it is designed using the power received over the power supply coupling 108. When the electronic device 106 is an implant for releasing metered doses of medication, for instance, a metered dose of medication is released. Alternately, the electronic device 106 stimulates bodily tissue (e.g., nerves), monitors specific biochemical conditions, and so forth. Although this method step is described with reference to operations performed by biomedical implants, the operations for some implants may correspond to non-medical functionality, such as location tracking, data storage/communication, personal information access, and so on.

FIG. 6 illustrates an example method 600 of in-body power harvesting from flowing fluid using a disk generator. In the following discussion, power harvester 104 or other entities of the example environment 100 may provide means for implementing one or more of the operations described.

At 602, the method includes rerouting a fluid flowing through the body into tubes on opposing sides of a disk generator. Before being rerouted to flow into the disk generator, the fluid flows naturally through tubes in the body. By way of example, the fluid flows through blood vessels, lymphatic vessels, or urinary system tubes of the person 102. Further, consider FIG. 4, which illustrates a disk generator configuration of the power harvester 104 in accordance with one or more aspects generally at 400. In this example, a fluid flowing through the person 102 is rerouted to flow into the tubes 406, 408 on the two opposing sides of the disk generator. This rerouting may be accomplished by surgically introducing components (such as one or more shunts) into the person 102 that are capable of carrying the fluid from naturally occurring tubes, into and through the disk generator, and then back into the naturally occurring tubes.

At 604, the method includes generating magnetic fields in the tubes to deflect charged particles of the fluid into generally opposing portions of space in respective flattened disk-shaped circular containers. In the continuing example, the fluid flowing into and through the disk generator includes positively and negatively charged particles. With regard to a top side of the disk generator illustrated in FIG. 4, the magnet 414 serves as a magnetic field generator and generates a magnetic field within the tube 406. A similarly configured magnet generates a magnetic field within the tube 408. The fluid generally flows into the circular containers 402, 404 via the tubes 406, 408 in the directions indicated by the arrows 410, 412.

Further, when the fluid flowing through the tube 406 passes through the magnetic field generated by the magnet 414, positively charged particles of the fluid are deflected in a first direction within the circular container 402, e.g., via Lorentz forces (or based on the Hall effect). This magnetic field also deflects negatively charged particles of the fluid flowing through the tube 406 in a second direction within the circular container 402 that is generally opposed to the first direction. For instance, the magnetic field may deflect positively charged particles outward toward outer portions of the circular container 402. The magnetic field may also deflect negatively charged particles inward toward a center of the circular container 402. The magnetic field generated for the bottom portion of the disk generator may deflect the charged particles to generally opposing portions of space in the circular container 404 in a similar manner.

At 606, the method includes using electrodes that circle the circular containers to harvest energy based on a difference in potential between the charged particles deflected into the generally opposing portions of space. By way of example, the inner electrodes 416, 418 and the outer electrodes 420, 422 are used to harvest energy from the fluid flowing through the disk generator, via the tube 406, and past the magnetic field generated by the magnet 414. Likewise, inner and outer electrodes of the circular container 404 are used to harvest energy from the fluid flowing through the disk generator, via the tube 408, and past its respective magnetic field. Further, these electrodes harvest energy based on a difference in potential created by deflecting positively and negatively charged particles to generally opposing portions of space in the circular containers 402, 404. For instance, the inner electrodes 416, 418 may form one end of a circuit, and the outer electrodes 420, 422 may form the other end of the circuit. The separated fluid flowing past these electrodes causes current to flow in the circuit, enabling energy harvesting.

At 608, the method includes feeding the fluid that exits the circular containers back into the body. In accordance with one or more aspects, the fluid exits the circular containers in a direction perpendicular to which the fluid flows into the disk generator. As discussed in relation to the method at 502, fluid rerouting may be accomplished by surgically introducing into the body components that are capable of carrying the fluid, such as one or more shunts. Here, such components are used to carry the fluid exiting the circular containers 402, 404 back into the naturally occurring tubes of the body, e.g., back into blood vessels, lymphatic vessels, urinary system tubes, and so forth.

At 610, the method includes converting the harvested energy into electrical power that is usable by an electronic device. By way of example, the converter 120 converts the energy harvested by the disk generator configuration of the power harvester 104 into electrical power usable by the electronic device 106. At 612, the method includes transferring the electrical power to the electronic device. By way of example, the power is transferred from the disk generator configuration of the power harvester 104 to the electronic device 106 via the power supply coupling 108. At 614, the method includes operating the electronic device using the electrical power. By way of example, the electronic device 106 carries out the functionality for which it was designed using the power received over the power supply coupling 108.

### System-on-Chip

FIG. 7 illustrates an example system-on-chip 700, which includes components capable of implementing aspects of in-body power harvesting using flowing fluids. The system-on-chip 700 may be implemented as, or in, any suitable electronic device, such as implants to release metered doses of medication, implants to stimulate bodily tissue (e.g., nerves), implants to monitor specific biochemical conditions, and so on, or any other device that may utilize power harvested by an in-body power harvester leveraging flowing ionic fluids in the body to generate power.

The system-on-chip 700 may be integrated with, a microprocessor, storage media, I/O logic, data interfaces, logic gates, a transmitter, a receiver, circuitry, firmware, software, or combinations thereof to provide communicative or processing functionalities. The system-on-chip 700 may include a data bus (e.g., cross bar or interconnect fabric) enabling communication between the various components of the system-on-chip. In some aspects, components of the system-on-chip 700 may interact via the data bus to implement aspects of in-body power harvesting using flowing fluids.

In this particular example, the system-on-chip 700 includes processor cores 702, system memory 704, and cache memory 706. The system memory 704 or the cache memory 706 may include any suitable type of memory, such as volatile memory (e.g., DRAM), non-volatile memory (e.g., Flash), and the like. The system memory 704 and the cache memory 706 are implemented as a storage medium, and thus do not include transitory propagating signals or carrier waves. The system memory 704 can store data and processor-executable instructions of the system-on-chip 700, such as operating system 708 and other applications. The processor cores 702 execute the operating system 708 and other applications from the system memory 704 to implement functions of the system-on-chip 700, the data of which may be stored to the cache memory 706 for future access. The system-on-chip 700 may also include I/O logic 710, which can be configured to provide a variety of I/O ports or data interfaces for inter-chip or off-chip communication.

The system-on-chip 700 also includes the converter 120 and implant-specific circuitry 712, which may be embodied separately or combined with other components described herein. For example, the converter 120 may be integral with the power supply coupling 108 or the power storage 122 as described with reference to FIG. 1. The implant-specific circuitry 712 can be implemented to carry out functionality specific to an implant, such as to release metered doses of medication, stimulate bodily tissue (e.g., nerves), monitor specific biochemical conditions, and so on.

The implant-specific circuitry 712 may also be integrated with other components of the system-on-chip 700, such as the cache memory 706, a memory controller of the system-on-chip 700, or any other signal processing, modulating/demodulating, or condition sections within the system-on-chip 700. The implant-specific circuitry 712 and other components of the system-on-chip 700 may be implemented as hardware, fixed-logic circuitry, firmware, or a combination thereof that is implemented in association with the I/O logic 710 or other signal processing circuitry of the system-on-chip 700.

Although subject matter has been described in language specific to structural features or methodological operations, it is to be understood that the subject matter defined in the appended claims is not necessarily limited to the specific features or operations described above, including not necessarily being limited to the organizations in which features are arranged or the orders in which operations are performed.

What is claimed is:

1. An apparatus for harvesting power from a body, the apparatus comprising:
a magnetic field generator configured to generate a magnetic field to deflect charged particles of a fluid flowing through the body in a space formed by a vessel, the charged particles including positively charged particles and negatively charged particles, the magnetic field deflecting the positively charged particles in a first direction within the space and the negatively charged particles in a second direction within the space, the second direction generally opposing the first direction; and
electrodes configured to harvest energy based on a difference in potential between the positively charged particles and the negatively charged particles, the harvested energy being convertible by a converter coupled to the electrodes into electrical power that is usable by an electronic device.

2. The apparatus as recited in claim 1, wherein the vessel extends along a major axis and the fluid flows generally in a direction along the major axis.

3. The apparatus as recited in claim 1, wherein the vessel is naturally-occurring in the body.

4. The apparatus as recited in claim 3, wherein the vessel is one of a blood vessel, a lymphatic vessel, or a urinary system vessel.

5. The apparatus as recited in claim 1, wherein the vessel is artificial and implanted in the body as part of a surgical procedure.

6. The apparatus as recited in claim 1, further comprising a constriction configured to constrict the vessel upstream from the magnetic field to accelerate the fluid flowing within the space.

7. The apparatus as recited in claim 1, wherein the magnetic field generator and the electrodes are positioned downstream from a natural constriction that constricts the vessel and accelerates the fluid flowing within the space.

8. The apparatus as recited in claim 1, wherein the electrodes are positioned along the vessel at a position that is downstream from a center of the magnetic field.

9. The apparatus as recited in claim 1, further comprising a power supply coupling configured to couple the electrodes to the electronic device to supply the electrical power to the electronic device.

10. The apparatus as recited in claim 1, wherein the fluid is one of blood, lymph, urine, or cerebrospinal fluid.

11. The apparatus as recited in claim 1, wherein the magnetic field generator comprises one or more magnets.

12. The apparatus as recited in claim 1, further comprising the converter configured to convert the harvested energy to the electrical power.

13. The apparatus as recited in claim 1, wherein the converter is configured to convert the harvested energy to the electrical power for use by the electronic device, at least in part, by increasing a voltage of the harvested energy to match a voltage used by the electronic device.

14. The apparatus as recited in claim 1, wherein the electronic device is an implant inserted into the body through a surgical procedure.

15. A method for harvesting power from fluid flowing in a body, the method comprising:
generating a magnetic field to deflect charged particles of a fluid flowing through a space in the body, the charged particles including positively and negatively charged particles, and the magnetic field deflecting the positively and negatively charged particles in generally opposing directions;
harvesting energy, via electrodes, from the flowing fluid based on a difference in potential between a portion of the fluid with the positively charged particles and a portion of the fluid with the negatively charged particles; and
converting the harvested energy with a converter coupled to the electrodes to electrical power that is usable by an electronic device within the body.

16. The method as recited in claim 15, wherein the space is formed by a vessel that extends along a major axis and the fluid flows generally in a direction along the major axis.

17. The method as recited in claim 16, further comprising constricting the vessel upstream from the magnetic field to accelerate the fluid flowing within the space.

18. The method as recited in claim 15, further comprising transferring the electrical power to the electronic device.

19. The method as recited in claim 18, wherein the electrical power is transferred over a wired coupling to the electronic device.

20. The method as recited in claim 18, wherein the electrical power is wirelessly transferred to the electronic device.

21. The method as recited in claim 15, further comprising surgically implanting in the body a magnetic field generator that generates the magnetic field, the electrodes, and the converter.

22. The method as recited in claim 15, further comprising operating the electronic device using the electrical power.

23. An apparatus for harvesting power from a body, the apparatus comprising:
two stacked disk-shaped circular containers centered substantially around a shared longitudinal axis;
two tubes that are arranged on opposing sides of the circular containers and are configured to feed fluid from the body into a respective circular container;
one or more magnetic field generators configured to generate magnetic fields that deflect positively and negatively charged particles of the fluid flowing through the tubes into generally opposing portions of space within the circular containers; and
inner and outer circular electrodes that are integral with the circular containers and are configured to harvest energy based on a difference in potential between the positively charged particles and the negatively charged particles in the generally opposing portions of the space.

24. The apparatus as recited in claim 23, further comprising one or more rerouting components configured to reroute the fluid from at least one naturally occurring tube of the body into the two tubes, through the circular containers, and back into a normal course of flow through the body.

25. The apparatus as recited in claim 23, wherein the one or more rerouting components comprise shunts implanted through a surgical procedure.

26. The apparatus as recited in claim 23, wherein the fluid exits the circular containers at a direction perpendicular to a direction in which the fluid enters via the two tubes.

27. A method for harvesting power from fluid flowing in a body, the method comprising:
rerouting the fluid flowing through the body into tubes on opposing sides of a disk generator, the tubes feeding respective disk-shaped circular containers of the disk generator;
generating magnetic fields in the tubes to deflect positively and negatively charged particles of the fluid to generally opposing portions of space in the respective circular containers;
harvesting energy, via electrodes that circle the circular containers, based on a difference in potential between the positively charged particles and the negatively charged particles in the generally opposing portions of the space; and
converting the harvested energy to electrical power that is usable by an electronic device in the body.

28. The method as recited in claim 27, further comprising feeding the fluid that exits the circular containers back into tubes of the body, the fluid exiting the circular containers in a direction perpendicular to which the fluid flows into the disk generator.

29. The method as recited in claim 27, further comprising:
transferring the electrical power to the electronic device; and
operating the electronic device using the electrical power.

30. The method as recited in claim 27, further comprising surgically implanting the disk generator into the body, including inserting one or more rerouting components to reroute the fluid from naturally occurring tubes of the body into the tubes on the opposing sides of the disk generator.
